(19) United States
(12) Patent Application Publication
(10) Pub. No.: US 2010/0094426 A1
(43) Pub. Date: Apr. 15, 2010

(54) HYBRID INTERVERTEBRAL SPINAL IMPLANT

(76) Inventors: Joseph A. Grohowski, Jr., Glens Falls, NY (US); Mark Walter, Greenwich, NY (US); Tracy M. MacNeal, Saratoga Springs, NY (US)

Correspondence Address: Zitzmann Consulting, LLC;c/o CPA Global, P.O. Box 52050, Minneapolis, MN, 55402, US

(21) Appl. No.: 12/578,446

(22) Filed: Oct. 13, 2009

Related U.S. Application Data
(60) Provisional application No. 61/105,244, filed on Oct. 14, 2008.

(51) Int. Cl. A61F 2/44; B23P 11/02

(52) U.S. Cl. 623/17.16; 29/453

(57) ABSTRACT

A spinal implant of hybrid construction. The implant includes both porous and radiolucent elements. In this manner, the implant allows for substantial fusing of vertebrae while simultaneously allowing for useful follow-on evaluations through imaging. Furthermore, in spite of the potentially differing material character of the porous and radiolucent elements, they may nevertheless be coupled together in an interlocking configuration such that the implant exhibits the behavior of a single unitary device.

### CROSS REFERENCE TO RELATED APPLICATION

This Patent Document claims priority under 35 U.S.C. §119(e) to U.S. Provisional Application Ser. No. 61/105,244 entitled Hybrid Fusion Cage with Improved Fixation, filed on Oct. 14, 2008, which is incorporated herein by reference in its entirety.

### FIELD

Embodiments described relate to biological implants. In particular, embodiments of intervertebral spinal implants for placement adjacent vertebrae are described in detail.

### BACKGROUND

Spinal implants are often employed to address and treat spinal disorders. For example, interspinous implants which attach to the exterior of the vertebrae may be used to address certain spinal disorders such as scoliosis or fractures. Alternatively, a spinal implant may be an intervertebral device that is utilized to replace a herniated or degenerative disc. Additionally, intervertebral spinal implants may be used in conjunction with interspinous implants, for example, where the fusion of multiple vertebrae is sought. Regardless, the intervertebral spinal implant in particular, occupies a relatively unique position in a literal sense. That is, this spinal implant is surrounded by bone of adjacent vertebrae. In fact, as a matter of structural soundness, a degree of bone ingrowth relative to the intervertebral spinal implant is generally sought.

In order to achieve bone ingrowth relative to intervertebral spinal implants, metals such as titanium, cobalt, stainless steel and others may be employed to make up the body of the implants. Each implant may be particularly sized, shaped, and configured of a given interconnected porosity to enhance bone ingrowth as indicated. Indeed, conventional bioactive agents may even be provided at surfaces of the implant to further promote bone ingrowth. All in all, porous metals such as those noted here may serve as sound and effective material choices for intervertebral spinal implants.

Unfortunately, porous metals such as those noted are not radiolucent. As such, x-ray and other conventional imaging techniques are relatively ineffective at providing information following surgical placement of the implant. For example, an x-ray of a patient with such a spinal implant following surgical placement is not an effective tool in confirming the degree or nature of bone and other growth relative to the implant. More specifically, structural soundness as determined by the degree of bone ingrowth into the implant may not be confirmed. Rather, the surgeon or monitoring physician is likely to see no more than a large void on the x-ray, which confirms no more than orientation of the implant to some minor degree.

Given the importance of follow-on monitoring of ingrowth relative to the spinal implant, alternative radiolucent materials are often chosen to make up the body of the implant. For example, in some situations a bone graft may be utilized as an intervertebral spinal implant. Thus, conventional follow-on imaging techniques may be utilized to monitor patient progress following surgery. That is, the degree of bone ingrowth and eventual fusion of the bone may be monitored and confirmed to ensure success of the implant over time. Unfortunately, the availability of bone material for grafts is limited. Additionally, actual and/or perceived risk of infection is often associated with the use of bone material.

Due to the radiolucency and structural challenges faced by above noted spinal implant materials, a radiolucent polymer-based material may be selected to form the implant. For example, polyetheretherketone (PEEK) is a common material selected in the manufacture of intervertebral spinal implants. PEEK and other similar materials such as polyetherketone (PEK), and polyetherketoneketone (PEKK), are almost entirely radiolucent and highly biocompatible. Therefore, these materials are a good option for the implant, particularly in terms of addressing post surgical monitoring issues. However, because they are radiolucent, metallic bead markers are embedded into the radiolucent body to allow radiolocation by the physician during and after surgery.

Unfortunately, while highly biocompatible in a general sense, such radiolucent polymer-based materials are non-porous. Indeed, from a manufacturing standpoint, there is presently no practical or cost-effective manner of inducing a controlled porosity throughout a radiolucent polymer-based implant. Thus, bone ingrowth and/or fusing of bone through the body of the implant is not attainable. To date, efforts to address this drawback have included providing the implant with unique shaping such as with a hollowed out interior and/or jagged tooth-like surfaces. However, these measures fail to provide bone apposition to the level afforded by metal implants made of titanium. As a practical matter, the physician and patient are presently left with the primary option of employing a metal based implant for which follow-on monitoring is extremely difficult.

### SUMMARY

An embodiment of an intervertebral spinal implant is provided. The implant includes a porous ingrowth promoting portion with a first surface for interfacing a vertebra of a patient's spine. Additionally, a radiolucent body of the implant is provided that is secured to the porous portion at a second surface that is substantially opposite the first.

### BRIEF DESCRIPTION OF THE DRAWINGS

FIG. 1 is a side view of an embodiment of a spinal implant of hybrid construction disposed at the intervertebral space between adjacent vertebra.

FIG. 2A is an exploded perspective view of one embodiment of the spinal implant of FIG. 1 revealing interlocking mechanics of hybrid material portions thereof.

FIG. 2B is a side cross-sectional view of the spinal implant of FIG. 2A in an assembled state.

FIG. 3A is a perspective view of another embodiment of the spinal implant of FIG. 1.

FIG. 3B is an exploded perspective view of the spinal implant of FIG. 3A revealing interlocking mechanics of hybrid material portions thereof.

FIG. 4 is an exploded perspective view of another embodiment of the spinal Implant of FIG. 1 revealing interlocking mechanics of hybrid material portions thereof.

FIG. 5 is a side cross-sectional view of a spinal implant taken from 5-5 of FIG. 3A.

FIG. 6 is an enlarged view of a surface region of the spinal implant taken from 6-6 of FIG. 3A.

FIG. 7 is a perspective view of yet another embodiment of a spinal implant of hybrid construction for intervertebral placement.

FIG. 8 is a flow-chart summarizing an embodiment of forming a spinal implant of hybrid construction for intervertebral placement.

### DETAILED DESCRIPTION

Embodiments are described with reference to certain configurations of spinal implants for intervertebral positioning. These may include spinal implants of unique surface design. For example, the surface may be of open and/or roughened porosity. Additionally, surfaces may include tooth-like projections to aid in initial fixation of the implant following intervertebral positioning. Furthermore, bioactive agents may be employed at surfaces of the implant to further encourage such ingrowth. Regardless, embodiments described herein include at least one porous portion to accommodate bone ingrowth which is secured to a radiolucent body. Thus, the implant may be referred to herein as of ‘hybrid’ construction comprising two or more materials.

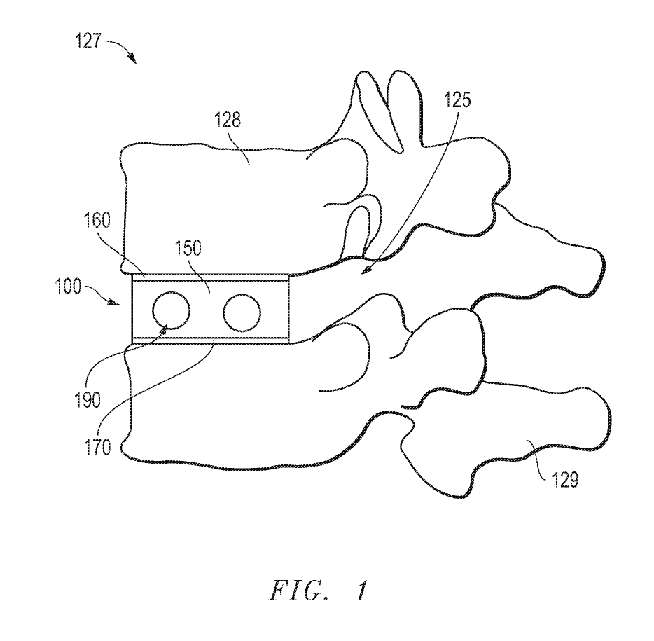

Referring now to FIG. 1, a portion of a patient's spine 127 is depicted. An embodiment of an intervertebral hybrid spinal implant 100 is shown implanted in the spine 127. More specifically, the implant 100 is disposed at the intervertebral space 125 between adjacent vertebrae 128, 129. Such positioning may be employed to maintain natural spacing between the vertebrae 128, 129, for example, where wear or injury has lead to the need for disc replacement. However, in an alternate embodiment, such an implant 100 may be configured for both disc and vertebral replacement. Regardless, in the embodiment shown, the implant 100 is configured to fuse the vertebrae 128, 129 together. As such, superior 160 and inferior 170 regions of the implant 100 are configured to serve as porous ingrowth promoting portions as detailed further below. Given that these regions 160, 170 may be of porous metal, the implant 100 also includes a radiolucent body 150. In this manner, follow-on imaging may be a viable manner of monitoring a patient's progress in attaining the noted implant stability. This may be particularly applicable where MRI is employed. That is, the reduction in metal may lead to a significant reduction in imaging artifacts. In addition, the porous metallic portion may be used for radiolocation, obviating the need of other types of metallic bead markers.

Continuing with reference to FIG. 1, the superior 160 and inferior 170 regions of the implant 100 may be of a porous biocompatible metal such as titanium or tantalum. The porous and roughened surface texture of such biocompatible metals of the implant 100 discourages its migration following placement. Such metals also exhibit significant bone apposition characteristics. Additionally, the porosity of these regions 160, 170 may be particularly tailored to promote ingrowth. Indeed, in one embodiment, conventionally available ingrowth promoting material may be accommodated at the surfaces of these regions 160 and throughout pores thereof to help stimulate bone ingrowth.

In other embodiments, the superior 160 and inferior 170 regions may be of alternate materials such as a nitride, carbide, or oxide of a porous metal. Additionally, a porous cobalt/chromium alloys or stainless steel may be used as the metal. In one embodiment, one or more of the regions 160, 170 may be constructed of a porous radiolucent material with a comparatively thin layer of metal such as titanium deposited thereover. Such a layer may itself be crystalline or amorphous in structure.

Given the generally radiographic incompatibility of porous metals, the body 150 of the implant 100 may be constructed of a radiolucent material such as a conventional biocompatible polymer. In one such embodiment, polyetheretherketone (PEEK) is employed as the material of the body 150. Additionally, in the embodiment shown, the radiolucent body 150 constitutes the majority of the side surface of the implant as shown, for example, from vertebra 128 to vertebra 129. Thus, a side x-ray image of the spine 127 in the area of the implant 100 will provide illustration of the majority of the area with only minority of image blocking by the superior 160 and inferior 170 regions. So, for example, bone ingrowth into and through these regions 160, 170 may be monitored in a practical manner.

Additionally, the radiolucency of the body 150 may be tailored to enhance imaging results. For example, in one embodiment, barium sulfate or another conventional contrast may be incorporated into the PEEK makeup of the body 150. In this manner, the radio-opacity may be provided to the body 150 in a visually perceptible manner upon imaging. So, for example, the orientation of the body 150 may be more directly determined. By the same token, imaging of the above noted regions 160, 170 may be employed to reveal the orientation of the overall implant 100 itself.

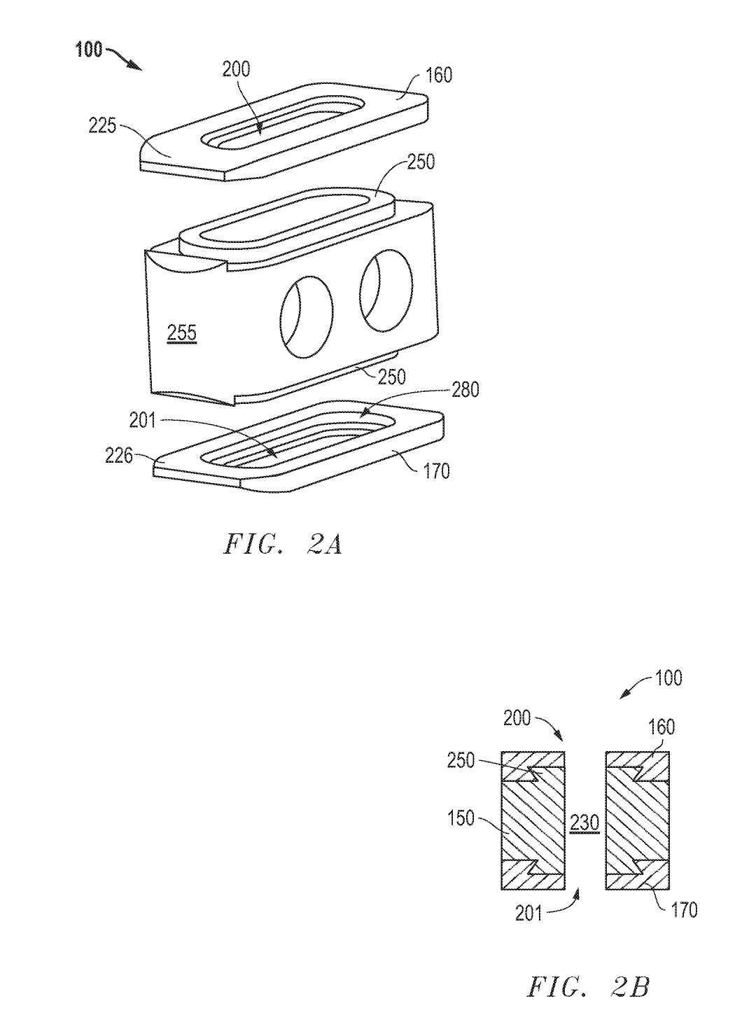

With brief added reference to FIGS. 2A, 2B, 3A and 4, differing implant configurations may be cage-like in nature as provided by the radiolucent body 150 to promote ingrowth as described above. In this manner, the implant 100 may actually be viewed as being of a cage-like nature, supporting ingrowth thereinto and ultimately vertebral fusion as described herein. Additionally, lateral openings 190 may be present through the body 150 in order to provide access to the noted internal space 230. As such, surgical access to the interior of the implant 100 may be provided. In one embodiment, bone or bone growth promoting material may be positioned at the internal space 230 prior to placement of the implant 100.

Referring now to FIGS. 2A and 2B more specifically, exploded and cross-sectional views of one embodiment of the spinal implant 100 are depicted. With respect to the exploded perspective view of FIG. 2A, the cage-like nature of the implant 100 is readily visible. Indeed, a superior opening 200 is present through the superior region 160 leading to the internal space 230 at the interior of the implant 100 (see FIG. 2B). A similar inferior opening 201 through the inferior region 170 may also be present. Regardless, access to the interior of the ‘cage’ may be quite extensive when considering all of the openings (190, 200, 201) throughout the body 150 and regions 160, 170 of the implant 100. Thus, ingrowth may be substantially allowed for.

Continuing with reference to FIG. 2A, each region 160, 170 is equipped with a tapered leading edge 225, 226. A nose 255 of the body 150 is configured to receive these edges 225, 226. Additionally, the tapered and rounded nature provided to this end of the implant 100 enhances the ability of the implant 100 to attain the positioning at the intervertebral space 125 as shown in FIG. 1.

In the embodiment of FIGS. 2A and 2B, the body 150 may also be equipped with tapers or protrusions 250 that are configured to be received by recesses 280 of the regions 160, 170. The protrusions 250 may be intentionally oversized relative to the recesses 280 and/or angled outward as revealed in the cross-sectional view of FIG. 2B. In this manner, fitting of the protrusions 250 into the recesses 280 may result in a secure and stable interlocking as described further below. Indeed, interlocking stresses may be kept at a minimum through use of such an embodiment. Nevertheless, the orientations of the protrusions 250 relative to the recesses 280 are such that the minimal compressive forces which are exerted in the axial direction are sufficient for drawing and holding the body 150 and the regions 160, 170 together.

Where the protrusions 250 are oversized in order to achieve interlocking as described above, the degree of oversizing may vary. For example, when viewing the body 150 from above and looking down on the protrusions 250, they may be oversized by between about 0.002 and 0.004 inches width-wise, and by between about 0.004 and 0.006 inches length-wise. In such an embodiment, the body 150 may be cooled to induce a reduction in size, thereby allowing the protrusions 250 to be received by the recesses 280. Later, the body 150 may be allowed to return to room temperature, increasing in size. Thus, compressive forces as noted above may be imparted at the interface of the body 150 and the regions 160, 170, thereby even more securely coupling these different elements to one another. Laser welding, heat staking, and/or adhesives may also be employed at the interface to enhance fastening of the body 150 and regions 160, 170. Additionally, in a related alternative embodiment, the noted regions 160, 170 may be configured to snap or press fit to the body 150.

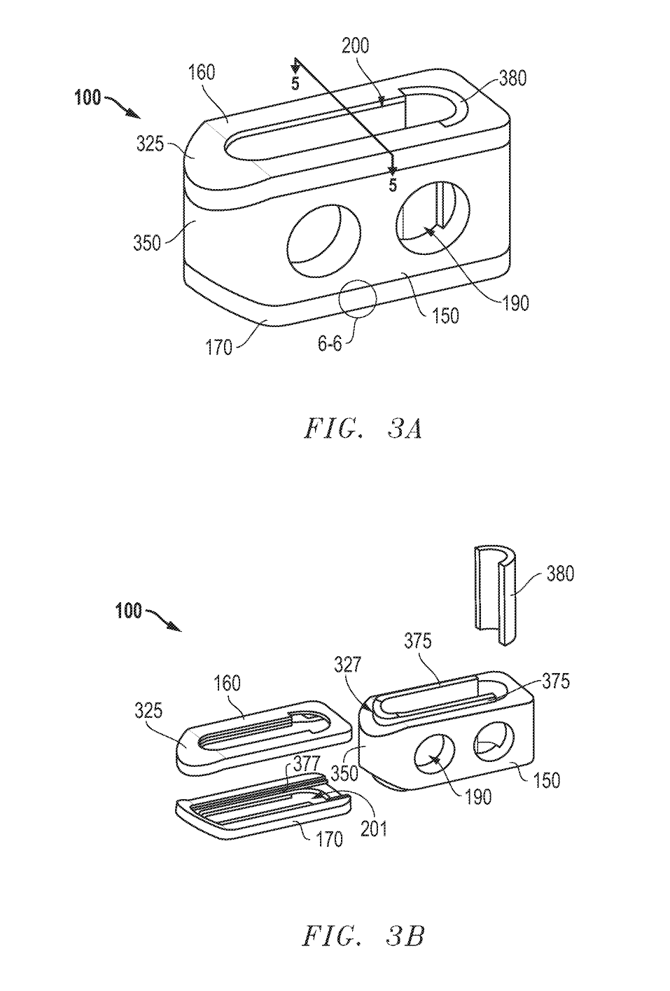

As depicted in FIG. 3A, an alternate embodiment of the implant 100 may be employed. In this embodiment, a retaining lock 380 (e.g. as opposed to oversizing) is employed to achieve stable interlocking between the body 150 and the regions 160, 170. Regardless, the use of such interlocking embodiments allows for a reduction in compressive stresses translated through the interface of the body 150 and the regions 160, 170. So, for example, such interlocking embodiments may be of an overall tensile strength sufficient for withstanding compression testing that subjects the implant 100 to a peak load of 500 lbs. at a frequency of 10 Hz for up to 5 million cycles or more without failure.

As alluded to above and detailed further below, the implant 100 may be of an interlocking configuration such that the porous metal regions 160, 170 slidably secured to the polymeric body 150 and held in place by the retaining lock 380. However, in alternate embodiments, the body 150 may be roughened or textured by way of media blasting, sanding, brushing, texture cutting or other conventional technique followed by application of a biocompatible adhesive for securing porous metal portions 160, 170, in place. The adhesive may be a bone, cyanoacrylate, or acrylic based cement. Additionally, the viscosity of the adhesive may be tailored to avoid any significant capillary flow into the porous metal material of the noted portions 160, 170. For example, in the case of bone cement, sufficient polymer powder may be mixed with monomer liquid to avoid such capillary action.

Continuing with reference to FIG. 3B, an exploded perspective view of the spinal implant 100 of FIG. 3A is shown. In this view, the hybrid material nature of the implant 100 is apparent. That is, the polymeric body 150 is shown separated from the porous metal regions 160, 170. Nevertheless, once securely assembled as described below, the implant 100 may behave in a unitary fashion as a cohesive intervertebral fusion device as depicted in FIG. 1.

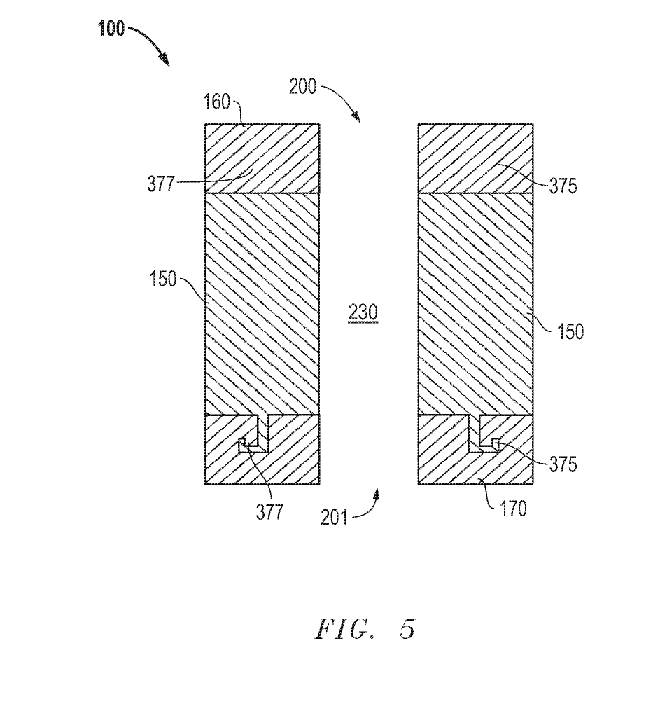

As indicated, the implant 100 may be hybrid in nature with separate features made up of different material types, such as the superior 160 and inferior 170 regions as compared to the body 150. Therefore, measures may be taken in order to ensure that the implant 100 retains a naturally unitary form. As shown in the embodiment of FIG. 3B, the implant 100 may again be of an interlocking character. That is, in this particular embodiment tracks 375 of the body 150 are provided to interlockingly pair with mating portions 377 of the regions 160, 170. Thus, the superior region 160 for example, may be slid over the tracks 375 until the tapered edge 325 reaches an abutment 327 of the body 150. Once both regions 160, 170 have been engaged with the body 150 in this manner, the retaining lock 380 may be inserted through the openings 200, 201 as shown, thereby holding the regions in place. This type of tracking engagement is also described in further detail below with reference to FIG. 5.

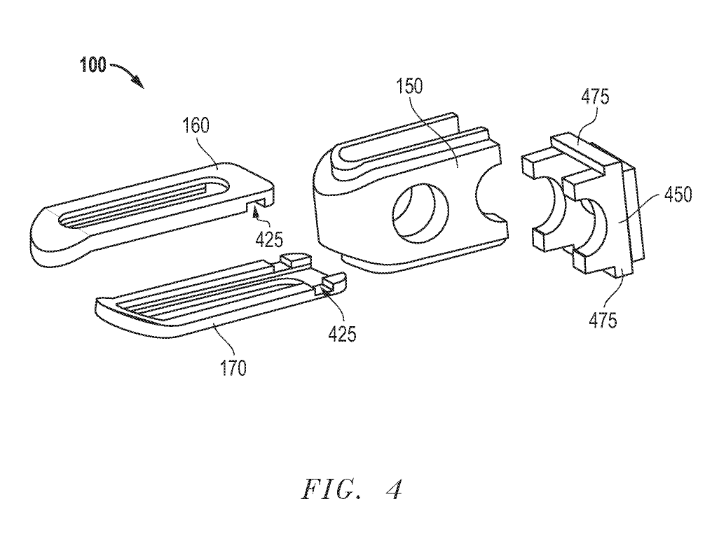

Referring now to FIG. 4, another alternate mechanism for interlocking of the regions 160, 170 relative to the body 150 is depicted. In this embodiment, the retaining lock 380 of FIGS. 3A and 3B is substituted with a locking end cap 450. That is, as opposed to sliding a lock 380 through the openings 200, 201 as depicted in FIG. 3, the implant 100 is equipped with an end that is defined by a lock 450. In particular, the locking end cap 450 is equipped with extensions 475 that are configured to be received by channels 425 of the regions 160, 170 once slid over the body 150 as described above. Indeed, any number of interlocking configurations may be employed with track-like embodiments such as those of FIGS. 3A, 3B, and 4, including with or without locks 380, 450.

Referring now to FIG. 5, a side cross-sectional view of the implant 100 is shown taken from 3-3 of FIG. 3A. In this view, the interlocking relationship of the superior 160 and inferior 170 regions relative to the body 150 are notably visible. More specifically, the tracks 375 which protrude from the body 150 are shown interlockingly engaged with the mating portions 377 of each region 160, 170. In this manner, a secure and stable fit between different features of the implant 100 which may be of vastly different material character may be stably attained. Thus, a substantially unitary spinal implant 100 may be provided.

As shown in FIG. 5, superior 200 and inferior 201 openings are also provided. These openings 200, 201 lead to an internal space 230 which, as indicated above, may or may not accommodate bone and/or other osteoinductive media. Regardless, over time, bone ingrowth through the porous superior 160 and inferior 170 regions and toward the internal space 230 may be achieved.

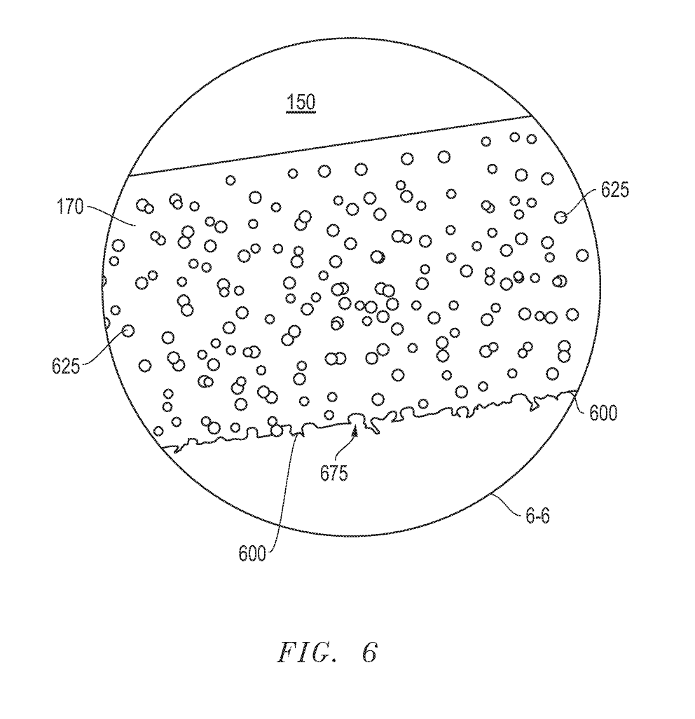

Referring now to FIG. 6, with added reference to FIG. 3A, an enlarged view of the implant 100 is shown taken from 6-6. In this depiction, the roughened surface 600 of the inferior region 170 is apparent. In one embodiment a calcium phosphate based ceramic may be coated on this surface 600 to help promote bone ingrowth. Other osteointegration promoting substances may also be employed in a similar manner.

Also apparent in the depiction of FIG. 6 is the porous nature of the region 170. Indeed, unlike the body 150 of the implant 100, interconnected pores 625 are apparent throughout the inferior region 170. In the embodiment shown, the major pore diameter is between about 70 and 500 microns whereas the minor pore diameter is between about 40 and about 225 microns. Additionally, pore shape may be spherical, cubic or irregular and the overall porosity may range from about 10 to about 800 microns. In one embodiment, each region 160, 170 is of a density that is less than about 55% (e.g. more than about 45% porosity). More particularly, in an embodiment where the regions 160, 170 are of titanium, the density may be less than about 50% with a compressive strength of at least about 25 MPa.

The porosity of the region 170 particularly adds to the roughened nature of the surface 600 where open pores 475 may be present. The keeled or serrated rough surface 600 of the inferior region 170 may be present at the superior region 160 as well (see FIG. 3A). Additionally, this roughening may be enhanced by way of micro-texturing or other techniques that extend the overall roughness to between about 150 to 250 microns into the surface 600 in one embodiment. However, in other embodiments, texturing may extend the roughness up to about 500 microns into the surface 400.

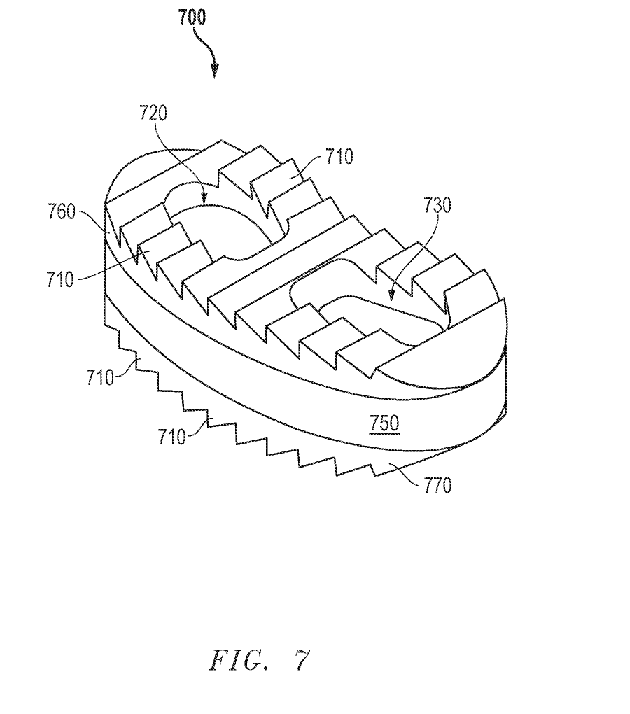

Referring now to FIG. 7, a perspective view of another alternate embodiment of spinal implant 700 is depicted. In this embodiment, the implant 700 is again of hybrid construction and configured for intervertebral placement. However, unlike the more block-shaped configuration depicted in FIGS. 1-6, the implant 700 of FIG. 7 is of an overall arcuate or banana-shaped configuration to suit a correspondingly shaped intervertebral space of a patient. Additionally, each end of the implant 700 is substantially rounded off. Along these same lines, alternate embodiments of the implant 700 may be of a horseshoe, circular, or vertebral-shaped morphological configuration depending on the implant application and the nature of the intervertebral space.

In addition to the differing overall shape, the implant 700 is equipped with multiple openings 720, 730. These openings 720, 730 traverse the superior 760 and inferior 770 regions as well as the body 750 therebetween. Thus, the space to accommodate bone or other biocompatible or even ingrowth promoting material is provided. Additionally, each region 760, 770 is equipped with teeth 710 to help immobilize the implant 700 from the very initial placement at the intervertebral space.

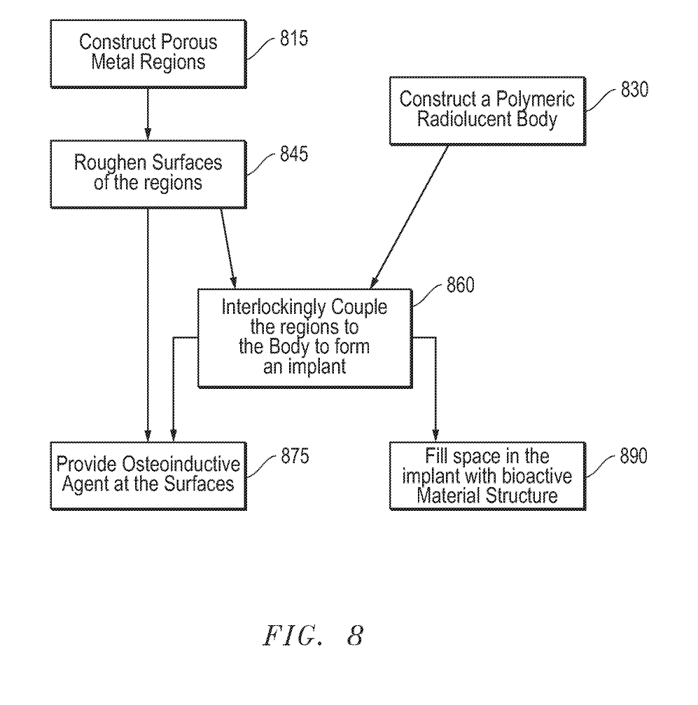

Referring now to FIG. 8, a flow-chart summarizing an embodiment of forming a spinal implant of hybrid construction is shown. As indicated above, separate porous metal regions and related features may be formed along with a polymeric radiolucent body (see 815, 830). More specifically, the polymeric body may be formed via injection mold or other conventionally available techniques. Additionally, the porous metal regions may be formed of a particularly tailored pore character as detailed in U.S. application Ser. No. 10/884,444, for Porous Metal Articles Having a Predetermined Pore Character. Additionally, alternate pore forming techniques may be employed. As indicated at 845, and described hereinabove, surfaces of the porous metal regions may be roughened. This may be done at surfaces likely to come into contact with bone. As such, the roughening at the surface may help to promote ingrowth relative to the porous metal region.

Once available, the radiolucent body and the porous metal regions may be formed into a single hybrid implant device. As indicated at 860, the coupling may take place via interlocking as detailed hereinabove. In this manner, challenges inherent to employing substantially different material types may be avoided. For example, reliance on joining techniques such as ultrasonic bonding, injection molding, solvent welding, and laser welding, which may work well with one material type to the exclusion of the other may be replaced with interlocking as described above. Furthermore, additional measures may similarly be taken to help ensure the unitary behavior of the implant as a whole in spite of the utilization of multiple material types of differing character. For example, while not required, biocompatible adhesives may be employed at interfacing of the polymeric body and the porous metal regions.

To further enhance the structural and biological compatibility of the implant, an osteoinductive agent may be provided at the surfaces of the porous metal regions as indicated at 875. Such agent may be added before or after coupling of the metal regions to the polymeric body of the implant. Additionally, internal space of the body may be filled with bioactive material structure such as bone or graft material as indicated at 890.

Embodiments described herein provide for a spinal implant that is both substantially radiolucent while at the same time having a porosity at surfaces thereof that are configured for interfacing bone. As such, imaging may be substantially enhanced while simultaneously encouraging bone ingrowth and structural soundness between the implant and vertebrae of a patient.

The preceding description has been presented with reference to presently preferred embodiments of the invention. Persons skilled in the art and technology to which this invention pertains will appreciate that alterations and changes in the described structures and methods of operation can be practiced without meaningfully departing from the principle, and scope of this invention. Regardless, the foregoing description should not be read as pertaining only to the precise structures described and shown in the accompanying drawings, but rather should be read as consistent with and as support for the following claims, which are to have their fullest and fairest scope.

We claim:

1. A spinal implant for positioning at an intervertebral space and comprising:
a porous portion having a first surface for interfacing a vertebra defining the intervertebral space; and
a radiolucent body coupled to said porous portion at a second surface thereof, substantially opposite the first surface.

2. The spinal implant of claim 1 wherein said radiolucent body is coupled to said porous portion through interlocking engagement.

3. The spinal implant of claim 1 wherein said porous portion is metal.

4. The spinal implant of claim 3 wherein the metal is one of titanium, titanium alloy, cobalt/chromium alloy, tantalum, and stainless steel.

5. The spinal implant of claim 3 wherein said porous portion is one of a nitride, a carbide, and an oxide of the metal.

6. The spinal implant of claim 1 wherein said porous portion is a metal coated radiolucent material.

7. The spinal implant of claim 6 wherein the metal comprises titanium.

8. The spinal implant of claim 1 wherein said porous portion is one of a superior porous portion for interfacing the vertebra at a superior position relative to the intervertebral space and an inferior porous portion for interfacing the vertebra at an inferior position relative to the intervertebral space.

9. The spinal implant of claim 1 wherein said porous portion comprises pores having a major pore diameter of between about 70 microns and about 500 microns.

10. The spinal implant of claim 1 wherein said porous portion comprises pores having a minor pore diameter of between about 40 microns and about 225 microns.

11. The spinal implant of claim 1 wherein said porous portion has a porosity of more than about 45%.

12. The spinal implant of claim 1 wherein said porous portion has a compressive strength of at least about 25 MPa.

13. The spinal implant of claim 1 wherein said radiolucent body is of a cage-like configuration to accommodate bone material at an internal space thereof.

14. The spinal implant of claim 1 wherein said radiolucent body is a biocompatible polymer.

15. The spinal implant of claim 14 wherein the biocompatible polymer is polyetheretherketone.

16. The spinal implant of claim 14 wherein the biocompatible polymer includes an imaging contrast incorporated therein.

17. An intervertebral implant for positioning at a spine and comprising:
a radiolucent body; and
a porous portion for interlocking engagement with said radiolucent body at one side thereof and configured for interfacing bone of the spine at a substantially opposite side thereof.

18. The intervertebral implant of claim 17 wherein said radiolucent body comprises tracks extending therefrom to slidably receive mating portions extending from said porous portion to allow for the engagement.

19. The intervertebral implant of claim 17 wherein said radiolucent body is of a biocompatible polymer and said porous portion is of metal.

20. The intervertebral implant of claim 19 wherein said porous portion is of a size to be used for radiolocation without obstructing the biocompatible polymer.

21. A spinal implant comprising:
a superior porous metal portion having a first surface for interfacing a superior vertebra defining a superior side of an intervertebral space;
a polymeric radiolucent body coupled to a second surface of said superior porous metal portion substantially opposite the first surface; and
an inferior porous metal portion having a first surface for interfacing an inferior vertebra defining an inferior side of the intervertebral space and a second opposite surface coupled to said polymeric radiolucent body.

22. The spinal implant of claim 21 having a height of between about 5 mm and about 15 mm.

23. The spinal implant of claim 21 having a length of up to about 30 mm.

24. The spinal implant of claim 21 wherein each of said porous metal portions is of a height between about 0.75 mm and about 1.75 mm.

25. The spinal implant of claim 21 having a shape that is substantially one of horseshoe, circular, banana, block, and vertebral.

26. The spinal implant of claim 21 wherein the first surfaces comprise teeth.

27. The spinal implant of claim 21 wherein the first surfaces are of a roughness extending between about 150 microns and about 250 microns thereinto.

28. The spinal implant of claim 21 further comprising a coating of a calcium phosphate based ceramic at the first surfaces to promote vertebral bone ingrowth thereinto.

29. A method of forming a spinal implant for intervertebral placement, the method comprising interlockingly coupling a porous metal portion to a polymeric radiolucent body.

30. The method of claim 29 wherein said coupling further comprises snap fitting the porous metal portion on the polymeric radiolucent body.

31. The method of claim 29 wherein said coupling further comprises:
cooling of the polymeric radiolucent body from an oversized state into fitting engagement with the porous metal portion at an interface thereof; and
returning the polymeric radiolucent body to the oversized state to impart substantial compressive force at the interface.

32. The method of claim 29 further comprising applying an adhesive at a surface of one of the porous metal portion and the polymeric radiolucent body prior to said coupling.

33. The method of claim 32 wherein the adhesive is a cement of one of bone, cyanoacrylate, and acrylic.

34. The method of claim 32 wherein the adhesive is of a tailored viscosity to avoid significant capillary uptake into the porous metal portion.

35. The method of claim 32 further comprising texturing of the surface by one of blasting, sanding, brushing, and cutting prior to said applying.

36. The method of claim 29 further comprising:
roughening a surface of the porous metal portion; and
providing an osteoinductive agent at the surface to promote vertebral growth thereinto.
